SEQUENCE LISTING (1 sequence)

SEQ ID NO: 1 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

(19) United States
(12) Patent Application Publication
(10) Pub. No.: US 2026/0001949 A1
(43) Pub. Date: Jan. 1, 2026

(54) NEUTRALIZATION OF ACYL-COA BINDING PROTEIN FOR THE TREATMENT OF CARDIAC DYSFUNCTION

(71) Applicants: INSERM (INSTITUT NATIONAL DE LA SANTÉ ET DE LA RECHERCHE MÉDICALE), PARIS (FR); UNIVERSITÉ PARIS CITÉ, Paris (FR); SORBONNE UNIVERSITÉ, PARIS (FR); ASSISTANCE PUBLIQUE-HÔPITAUX DE PARIS, PARIS (FR); UNIVERSITÉ PARIS-SACLAY, GIF-SUR-YVETTE (FR); INSTITUT GUSTAVE ROUSSY, Villejuif (FR)

(72) Inventors: Guido KROEMER, Paris (FR); Mahmoud ABDELLATIF, PARIS (FR); Léa MONTÉGUT, PARIS (FR); Isabelle MARTINS, PARIS (FR)

(21) Appl. No.: 18/881,529

(22) PCT Filed: Jul. 21, 2023
(86) PCT No.: PCT/IB2023/057473

§ 371 Date: Jan. 6, 2025

(30) Foreign Application Priority Data
Jul. 22, 2022 (EP) 22306096.3

(51) Int. Cl. C07K 16/26; A61P 9/10

(52) U.S. Cl. CPC C07K 16/26; A61P 9/10

(57) ABSTRACT

Cardiomyopathies are heart muscle disorders which represent a heterogeneous group of diseases that often lead to progressive heart failure with significant morbidity and mortality. Here, the inventors show that neutralization of acyl CoA binding protein (ACBP)/diazepam binding inhibitor (DBI) reduces the deleterious effect of the Vehicle anthracycline doxombicin on cardiac function. Since anthracycline-induced heart failure and anthracycline-induced senescence are models of accelerated cardiac aging, it is also plausible to use ACBP/DBI inhibition or neutralization as a method to prevent or treat aging of the cardiovascular system. The inventors also found that genetic and pharmacological interventions to deplete ACBP/DBI efficiently improves cardiac dysfunction associated with experimental HFpEF, a condition associated with aging, hypertension and obesity. Accordingly, the present disclosure relates to methods for the treatment of cardiac dysfunction comprising neutralization of ACBP/DBI.

### CROSS REFERENCE TO RELATED APPLICATIONS

This application claims priority to European Application Serial No. EP 22306096, filed 22 Jul. 2022, the disclosure of which is incorporated herein by reference in its entirety.

### INCORPORATION BY REFERENCE OF SEQUENCE LISTING

The instant application contains a Sequence Listing, which has been submitted via Patent Center. The Sequence Listing titled 206008-704601_SL.xml, which was created on Jul. 21, 2023 and is 1,803 bytes in size, is hereby incorporated by reference in its entirety.

### TECHNICAL FIELD

The present disclosure relates to the field of medicine, for example the field of cardiology.

### BACKGROUND

Cardiomyopathies are heart muscle disorders which represent a heterogeneous group of diseases that often lead to progressive heart failure with significant morbidity and mortality. Common symptoms include dyspnea and peripheral oedema and risks of having dangerous forms of irregular heart rate and sudden cardiac death are increased. For instance, heart failure with preserved ejection fraction (HFpEF) is currently the predominant form of heart failure and the leading cause of hospitalization in the elderly. However, limited understanding of the underlying mechanisms of HFpEF has led to a longstanding absence of evidence-based therapies. In this respect, growing epidemiological and experimental evidence suggests that metabolic dysfunction might drive the pathogenesis of HFpEF. In fact, the majority of HFpEF patients are obese or diabetic, suggesting that metabolic therapies acting on the heart and peripheral organs are worth considering.

Acyl coenzyme A binding protein (ACBP), which is encoded by diazepam-binding inhibitor (DBI), is a leaderless polypeptide that can be secreted in an unconventional fashion during the activation of autophagy. However, the role of ACBP in cardiomyopathies has never been investigated.

### BRIEF SUMMARY

The present disclosure is defined by the claims. For instance, the present disclosure relates to methods for the treatment of cardiac dysfunction comprising neutralization of acyl-CoA binding protein (ACBP) (also referred to as diazepam binding inhibitor (DBI)).

Disclosed herein are methods of treating cardiac dysfunction in a patient in need thereof, the method comprising administering a therapeutically effective amount of an agent that reduces the activity or expression of diazepam binding inhibitor (DBI) to the patient. In some embodiments, the cardiac dysfunction comprises ischemic cardiomyopathy, myocardial ischemia, myocardial infarction, or any cardiac condition associated with limited oxygen availability or reduced cell viability. In some embodiments, the agent improves cardiac remodeling, cardiac fibrosis, impairment of systolic function, or impairment of diastolic dysfunction, each as compared to prior to the administering. In some embodiments, the patient is an elderly patient, an obese patient, or a patient who exhibits one or more risk factors for cardiac dysfunction. In some embodiments, the one or more risk factors for cardiac dysfunction is chosen from age, alcohol consumption, cigarette smoking, metabolic syndrome, obesity, diabetes/insulin resistance, hypertension, dyslipidemia, liver disease and chronic kidney disease. In some embodiments, the patient suffers from a cardiomyopathy. In some embodiments, the cardiomyopathy is dilated cardiomyopathy, metabolic cardiomyopathy, ischemic cardiomyopathy, hypertrophic cardiomyopathy, non-obstructive cardiomyopathy, restrictive cardiomyopathy, left ventricular non-compaction, or arrhythmogenic right ventricular cardiomyopathy. In some embodiments, the cardiomyopathy is from one or more of the following non familial causes: obesity, a diabetic mother, athletic training, myocarditis, Kawasaki disease, an eosinophilic disease, viral persistence, pregnancy, endocrine dysfunction, a nutritional imbalance, alcohol, tachycardiomyopathy, inflammation, amyloid dysfunction, scleroderma, endomyocardial fibrosis, hypereosinophilic syndrome, a drug, carcinoid heart disease, a metastatic cancer, an antineoplastic drug, a psychiatric drug, chloroquine, all-trans retinoic acid, an anti-retro viral agent, and a phenothiazine. In some embodiments, the myocarditis is infective, toxic or immune myocarditis. In some embodiments, the eosinophilic disease is churg strauss syndrome. In some embodiments, the nutritional imbalance is chosen from a thymine nutritional imbalance, a carnitine nutritional imbalance, a selenium nutritional imbalance, hypophosphataemia, or hypocalcaemia. In some embodiments, the amyloid dysfunction is AL/prealbumin. In some embodiments, the patient suffers from a metabolic cardiomyopathy, wherein the metabolic cardiomyopathy is an age-related metabolic cardiomyopathy. In some embodiments, the cardiomyopathy is from drugs chosen from serotonin, methysergide, ergotamine, mecurial agents, or busulfan. In some embodiments, the antineoplastic drugs are chosen from anthracyclines, antimetabolites, alkylating agents, taxol, hypomethylating agents, monoclonal antibodies, tyrosine kinase inhibitors, and immunomodulating agents. In some embodiments, the psychiatric drugs are chosen from clozapine, olanzapine, chloropromazine, risperidone, lithium methylphenidate, and tricyclic antidepressants. In some embodiments, the patient suffers from heart failure. In some embodiments, the heart failure is a heart failure with preserved ejection fraction. In some embodiments, the patient suffers from myocardial infarction. In some embodiments, the agent that reduces the activity of DBI is an antibody or an aptamer directed against DBI. In some embodiments, the antibody is directed against a fragment of DBI consisting of amino acid residue 43 to the amino acid residue 50 of SEQ ID NO: 1. In some embodiments, the antibody is a monoclonal chimeric antibody, a monoclonal humanized antibody, or a monoclonal human antibody. In some embodiments, the agent that reduces the expression of DBI is an inhibitor of expression. In some embodiments, the inhibitor of expression is an siRNA, an endonuclease, an antisense oligonucleotide or a ribozyme. In some embodiments, the agent that reduces the activity of DBI is a vaccine composition suitable for eliciting neutralizing autoantibodies against DBI when administered to the patient. In some embodiments, the vaccine composition comprises a polypeptide antigen comprising (i) an amino acid sequence having at least 80% identity with SEQ ID NO: 1; (ii) an amino acid sequence having at least 80% identity to a polypeptide fragment consisting of the amino acid sequence ranging from amino acid residue 17 to amino acid residue 50 of SEQ ID NO: 1; (iii) an amino acid sequence having at least 80% identity to a polypeptide fragment consisting of the amino acid sequence ranging from amino acid residue 33 to amino acid residue 50 of SEQ ID NO: 1; or (iv) an amino acid sequence having at least 80% identity to a polypeptide fragment consisting of the amino acid sequence ranging from amino acid residue 43 to amino acid residue 50 of SEQ ID NO: 1. In some embodiments, the agent reduces the activity or expression of extracellular DBI. In some embodiments, the agent reduces the activity of extracellular DBI. In some embodiments, the agent reduces the activity of extracellular DBI by binding to extracellular DBI. In some embodiments, the binding to the extracellular DBI disrupts binding of the extracellular DBI to a binding partner naturally present in a human cell. In some embodiments, the binding partner is a gamma-aminobutyric acid type A receptor (GABR). In some embodiments, the agent binds to a surface of the extracellular DBI that is responsible for binding of the extracellular DBI to the GABR. In some embodiments, the agent reduces levels of extracellular DBI in circulation. In some embodiments, the agent reduces the activity of extracellular DBI, thereby resulting in induction of autophagy, wherein the induction of autophagy reduces levels of extracellular DBI in circulation. In some embodiments, the agent that reduces the levels of extracellular DBI in circulation does not reduce levels of intracellular DBI when administered to the patient.

Disclosed herein are methods of detecting and treating myocardial ischemia in a patient, the method comprising: (a) measuring a level of extracellular diazepam binding inhibitor (DBI) in a plasma sample obtained from the patient, wherein the level of extracellular DBI is determined in an in vitro immunoassay; (b) comparing the level of extracellular DBI in the patient to a reference level of extracellular DBI from a healthy subject; (c) diagnosing the patient as having myocardial ischemia when the level extracellular DBI in the patient is higher than the reference level; and (d) administering a therapeutically effective amount of an agent that reduces the activity or expression of extracellular DBI to the patient, thereby treating the myocardial ischemia.

Disclosed herein are compositions for use in treating cardiac dysfunction in a patient in need thereof, wherein the composition comprises a therapeutically effective amount of an agent that reduces the activity or expression of diazepam binding inhibitor (DBI). In some embodiments, the composition for use is for use in improving cardiac remodeling, cardiac fibrosis, impairment of systolic function, or impairment of diastolic dysfunction. In some embodiments, the composition for use is for treating cardiomyopathy. In some embodiments, the cardiomyopathy is ischemic cardiomyopathy. In some embodiments, the composition for use is for treating heart failure. In some embodiments, the composition for use is for treating myocardial infarction. In some embodiments, the agent reduces the activity or expression of extracellular DBI. In some embodiments, the agent reduces the activity of extracellular DBI. In some embodiments, the agent reduces levels of extracellular DBI in circulation. In some embodiments, the agent reduces the activity of extracellular DBI by binding to extracellular DBI. In some embodiments, the binding to the extracellular DBI disrupts binding of the extracellular DBI to a binding partner naturally present in a human cell. In some embodiments, the binding partner is a gamma-aminobutyric acid type A receptor (GABR). In some embodiments, the agent binds to a surface of the extracellular DBI that is responsible for binding of the extracellular DBI to the GABR. In some embodiments, the agent reduces the activity of extracellular DBI, thereby resulting in induction of autophagy, wherein the induction of autophagy reduces levels of extracellular DBI in circulation. In some embodiments, the agent that reduces the levels of extracellular DBI in circulation does not reduce levels of intracellular DBI. In some embodiments, the agent that reduces the activity or expression of DBI is an antibody or an aptamer directed against DBI. In some embodiments, the antibody is directed against a fragment of DBI consisting of amino acid residue 43 to the amino acid residue 50 of SEQ ID NO: 1. In some embodiments, the antibody is chosen from a monoclonal chimeric antibody, a monoclonal humanized antibody, or a monoclonal human antibody.

### FIGURES

FIGS. 1A-1G: ACBP neutralization attenuates anthracyline-induced cardiotoxicity. FIG. 1A is a schematic representation of ACBP-neutralizing antibody (anti-ACBP) administration to doxorubicin (DOX)-treated C57Bl/6J female mice. FIGS. 1B-1G represent pairwise comparisons between anti-ACBP-treated mice and their respective isotype (Iso)-treated controls using simple main effects of a factorial ANOVA including DOX and anti-ACBP as fixed factors. N=3-10 mice per groups, with P values shown on top of the panels in FIGS. 1B, 1C, 1D, 1E and 1G. In FIG. 1F, P values were calculated using a mixed model, including DOX, anti-ACBP and time as fixed factors. Bars and error bars show means and SEM, respectively, with individual data points superimposed. Abbreviations: echocardiography-derived left ventricular ejection fraction (LVEF); left ventricular end-diastolic volume normalized to body surface area (LVEDVᵢ); left ventricular mass index (LVmassᵢ), calculated as the ratio between LVmass and body surface area; tibia length-normalized lung weight (LW/TL); body weight (BW) gain; heart rate; beat per minute (bpm); and echocardiography (echo).

FIGS. 2A-2B: Senolytic activity of the ACBP-neutralizing antibody—Immunostaining of p21 in cardiac sections of doxorubicin-treated mice. Representative images of the left ventricular areas shown in FIG. 2A were stained for the nuclear senescence marker p21. FIG. 2B shows quantification of p21 in the left ventricular areas. Statistical significance was tested by two-way ANOVA with Tukey's correction for multiple comparisons.

FIGS. 3A-3G: Cardioprotection against HFpEF in ACBP knockout mice. As shown in FIG. 3A a high-fat diet (HFD) and N[ω]-nitro-1-arginine methyl ester (L-NAME) was administered to Acbp-expressing (Acbp+/+) and Acbp-deficient (Acbp−/−) male mice for 8 weeks, with the following echocardiography-derived parameters determined at week 0 (baseline) and week 8 (HFpEF): FIG. 3B—left ventricular ejection fraction (LVEF); FIG. 3C—left ventricular remodelling index (LVRI), calculated as the ratio of LV mass to internal diastolic diameter; FIG. 3D—ratio of peak early Doppler transmitral flow velocity (E) to myocardial tissue Doppler velocity (e′), a measure of diastolic dysfunction; FIG. 3E—cardiac output; FIG. 3F—body weight (BW); and FIG. 3G—body weight (BW) gain. N=6 mice per groups. P values on top of the panels in FIGS. 3B-3F represent factor comparisons by 2-way repeated-measures ANOVA including Genotype and the HFpEF protocol or Genotype and Time (FIG. 3G) as fixed factors, followed by simple main effects analysis of pairwise comparisons between Acbp+/+ and Acbp−/− mice. Bars and error bars show means and SEM, respectively, with individual data points superimposed.

FIGS. 4A-4G: Cardioprotection against HFpEF using an ACBP-neutralizing antibody. As shown in FIG. 4A high-fat diet (HFD) and N[ω]-nitro-1-arginine methyl ester (L-NAME) were administered to C57Bl/6J male mice (10-week-old), which were either treated with an ACBP-neutralizing antibody (anti-ACBP) or mouse isotype IgG (CTRL) for 8 weeks. Subsequently, the following echocardiography-derived parameters were determined: FIG. 4B—left ventricular ejection fraction (LVEF); FIG. 4C—left ventricular remodelling index (LVRI), calculated as the ratio of LV mass to internal diastolic diameter; FIG. 4D—ratio of peak early Doppler transmitral flow velocity (E) to myocardial tissue Doppler velocity (e′);

FIG. 4E—cardiac output; FIG. 4F—body weight (BW); and FIG. 4G—body weight (BW) gain. N=9-10 mice per groups. P values on top of the panels in FIGS. 4B-4F represent factor comparisons by a 2-way independent ANOVA including anti-ACBP treatment and the two-hit HFpEF model as fixed factors, followed by simple main effects analysis of pairwise comparisons between Anti-ACBP-treated mice and their respective isotype-treated controls. In FIG. 4G, P values were calculated using a mixed model, including HFpEF, Anti-ACBP and time as fixed factors. Bars and error bars show means and SEM, respectively, with individual data points superimposed.

### DETAILED DESCRIPTION

The first object of the present disclosure relates to a method of treating cardiac dysfunction in patients in need thereof comprising administering a therapeutically effective amount of an agent that inhibits the activity or expression of diazepam binding inhibitor (DBI), (also referred to herein as acyl coenzyme A binding protein (ACBP)). In some embodiments, the present disclosure provides for a method of treating cardiac dysfunction in a patient in need thereof, the method comprising administering a therapeutically effective amount of an agent that reduces the activity or expression of diazepam binding inhibitor (DBI) to the patient.

As used herein, the term “cardiac dysfunction” also referred to as “myocardial dysfunction” is known by the person skilled in the art. The term relates to any kind of heart dysfunction, more particularly, the term relates to heart dysfunction affecting the pumping capability of the heart. In particular, the term “cardiac dysfunction” relates to a condition in which myocardial contractility, metabolism and ventricular function are reduced in order to cope with a reduced oxygen supply. Typically, cardiac dysfunction involves cardiac remodeling and/or cardiac fibrosis and/or impairment of systolic function (left ventricular ejection fraction (LVEF) function or strain) and/or impairment of diastolic dysfunction. Cardiac dysfunction may be asymptomatic and can occur out of any heart failure symptoms.

In some embodiments, the cardiac dysfunction comprises ischemic cardiomyopathy, myocardial ischemia, myocardial infarction, or any cardiac condition associated with limited oxygen availability or reduced cell viability. In some embodiments, the cardiac dysfunction comprises ischemic cardiomyopathy. In some embodiments, the cardiac dysfunction comprises myocardial ischemia. In some embodiments, the cardiac dysfunction comprises myocardial infarction. In some embodiments, the cardiac dysfunction comprises any cardiac condition associated with limited oxygen availability. In some embodiments, the cardiac dysfunction comprises any cardiac condition associated with reduced cell viability. As used herein, the terms “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen” which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, the agent improves cardiac remodeling, cardiac fibrosis, impairment of systolic function, or impairment of diastolic dysfunction, each as compared to prior to the administering. In some embodiments, the agent improves cardiac remodeling as compared to prior to the administering. In some embodiments, the agent improves cardiac fibrosis as compared to prior to the administering. In some embodiments, the agent improves impairment of systolic function as compared to prior to the administering. In some embodiments, the agent improves impairment of diastolic dysfunction, each as compared to prior to the administering.

In particular, the therapeutic method of the present disclosure is thus particularly suitable for the treatment of cardiac dysfunction in elderly patients, and/or obese patients and/or patients who exhibit one or more risk factors for cardiac dysfunction (e.g., age, alcohol consumption, cigarette smoking, metabolic syndrome, obesity, diabetes/insulin resistance, hypertension, dyslipidaemia, liver disease or chronic kidney disease).

As used herein, the term “elderly patient” refers to an adult patient sixty-five years of age or older.

As used herein, the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

In some embodiments, the patient is an elderly patient, an obese patient, or a patient who exhibits one or more risk factors for cardiac dysfunction. In some embodiments, the patient is an elderly patient. In some embodiments, the patient is an obese patient. In some embodiments, the patient is a patient who exhibits one or more risk factors for cardiac dysfunction. In some embodiments, the one or more risk factors for cardiac dysfunction is chosen from age, alcohol consumption, cigarette smoking, metabolic syndrome, obesity, diabetes/insulin resistance, hypertension, dyslipidemia, liver disease and chronic kidney disease. In some embodiments, wherein the one or more risk factors for cardiac dysfunction is age. In some embodiments, the one or more risk factors for cardiac dysfunction is chosen from alcohol consumption. In some embodiments, the one or more risk factors for cardiac dysfunction is cigarette smoking. In some embodiments, the one or more risk factors for cardiac dysfunction is metabolic syndrome. In some embodiments, the one or more risk factors for cardiac dysfunction is obesity. In some embodiments, the one or more risk factors for cardiac dysfunction is diabetes/insulin resistance. In some embodiments, the one or more risk factors for cardiac dysfunction is hypertension. In some embodiments, the one or more risk factors for cardiac dysfunction is dyslipidemia. In some embodiments, the one or more risk factors for cardiac dysfunction is liver disease. In some embodiments, the one or more risk factors for cardiac dysfunction is chronic kidney disease. In some embodiments, the one or more risk factors is chosen from one, two, three, four, five, six, seven, eight, nine, or ten of the foregoing risk factors.

In some embodiments, the patient suffers from a cardiomyopathy.

As used herein, the term “cardiomyopathy” has its general meaning in the art and refers to any disease of the heart muscle. Cardiomyopathy can be acquired or inherited. In some embodiments, the cardiopathy is an non-ischemic cardiomyopathy, more particularly myocardial infarction. Common symptoms include dyspnea and peripheral oedema, and risks of having dangerous forms of irregular heart rate and sudden cardiac death are increased. Cardiomyopathy often leads to progressive heart failure, i.e., the incapacity of the cardiac pump to maintain sufficient blood flow to meet the basal bodily needs for oxygen. The main types of cardiomyopathy include dilated cardiomyopathy, hypertrophic cardiomyopathy, non-obstructive cardiomyopathy, restrictive cardiomyopathy, left ventricular non-compaction, and arrhythmogenic right ventricular cardiomyopathy. Dilated cardiomyopathy is characterized by dilatation and systolic dysfunction of the left or both ventricles. The ventricular walls become thin and stretched, compromising cardiac contractility and ultimately resulting in poor left ventricular function.

In some embodiments, the cardiomyopathy may derive from the following non familial causes: obesity, infants of diabetic mothers, athletic training, amyloid (al/prealbumin), myocarditis (infective/toxic/immune), Kawasaki disease, eosinophilic (churg strauss, syndrome), viral persistence, pregnancy, endocrine, nutritional (thiamine, carnitine, selenium, hypophosphataemia, hypocalcaemia), alcohol, tachycardiomyopathy, inflammation, amyloid (AL/prealbumin), scleroderma, endomyocardial fibrosis, hypereosinophilic syndrome, drugs (serotonin, methysergide, ergotamine, mercurial agents, busulfan), carcinoid heart disease, metastatic cancers, antineoplastic drugs (anthracyclines, antimetabolites, alkylating agents, taxol, hypomethylating agents, monoclonal antibodies, tyrosine kinase inhibitors, immunomodulating agents), psychiatric drugs (clozapine, olanzapine, chlorpromazine, risperidone, lithium, methylphenidate, tricyclic antidepressants) and other drugs such as chloroquine, all-trans retinoic acid, antiretroviral agents and phenothiazines. More particularly, the cardiomyopathy results from direct and indirect sympathomimetics, beta-blockers, anticholinergics, cholinomimetics, adenosine receptor agonist, Ca²⁺ channel blockers, Na⁺/K⁺ ATPase inhibitors, lysosomotropic agents, anticancer chemotherapeutics (such as cytarabine, cyclophosphamide, daunorubicin, docetaxel, epirubicin, paclitaxel, treosulfan and other anthracyclines, alkylating agents and taxanes), HER2-targeting antibodies (such as adotrastuzumab trastuzumab, pertuzumab and others), tyrosine kinase inhibitors (such as dobrafenib, lapatinib, sorafenib, sunitinib, ponatinib and others), proteasome inhibitors (such as bortezomib, karfilzomib and others), non-steroidal anti-inflammatory agents, ethanol, nicotine and smoking.

In some embodiments, the cardiomyopathy is dilated cardiomyopathy, metabolic cardiomyopathy, ischemic cardiomyopathy, hypertrophic cardiomyopathy, non-obstructive cardiomyopathy, restrictive cardiomyopathy, left ventricular non-compaction, or arrhythmogenic right ventricular cardiomyopathy. In some embodiments, the cardiomyopathy is dilated cardiomyopathy. In some embodiments, the cardiomyopathy is metabolic cardiomyopathy. In some embodiments, the cardiomyopathy is ischemic cardiomyopathy. In some embodiments, the cardiomyopathy is hypertrophic cardiomyopathy. In some embodiments, the cardiomyopathy is non-obstructive cardiomyopathy. In some embodiments, the cardiomyopathy is restrictive cardiomyopathy. In some embodiments, the cardiomyopathy is left ventricular non-compaction. In some embodiments, the cardiomyopathy is arrhythmogenic right ventricular cardiomyopathy.

In some embodiments, the cardiomyopathy is from one or more of the following non familial causes: obesity, a diabetic mother, athletic training, myocarditis, Kawasaki disease, an eosinophilic disease, viral persistence, pregnancy, endocrine dysfunction, a nutritional imbalance, alcohol, tachycardiomyopathy, inflammation, amyloid dysfunction, scleroderma, endomyocardial fibrosis, hypereosinophilic syndrome, a drug, carcinoid heart disease, a metastatic cancer, an antineoplastic drug, a psychiatric drug, chloroquine, all-trans retinoic acid, an anti-retro viral agent, and a phenothiazine. In some embodiments, the cardiomyopathy is from obesity. In some embodiments, the cardiomyopathy is from a diabetic mother. In some embodiments, the cardiomyopathy is from athletic training. In some embodiments, the cardiomyopathy is from myocarditis. In some embodiments, the cardiomyopathy is from Kawasaki disease. In some embodiments, the cardiomyopathy is from an eosinophilic disease. In some embodiments, the cardiomyopathy is from viral persistence. In some embodiments, the cardiomyopathy is from pregnancy. In some embodiments, the cardiomyopathy is from endocrine dysfunction. In some embodiments, the cardiomyopathy is from a nutritional imbalance. In some embodiments, the cardiomyopathy is from alcohol. In some embodiments, the cardiomyopathy is from tachycardiomyopathy. In some embodiments, the cardiomyopathy is from inflammation. In some embodiments, the cardiomyopathy is from amyloid dysfunction. In some embodiments, the cardiomyopathy is from scleroderma. In some embodiments, the cardiomyopathy is from endomyocardial fibrosis. In some embodiments, the cardiomyopathy is from hypereosinophilic syndrome. In some embodiments, the cardiomyopathy is from a drug. In some embodiments, the cardiomyopathy is from carcinoid heart disease. In some embodiments, the cardiomyopathy is from a metastatic cancer. In some embodiments, the cardiomyopathy is from an antineoplastic drug. In some embodiments, the cardiomyopathy is from a psychiatric drug. In some embodiments, the cardiomyopathy is from chloroquine. In some embodiments, the cardiomyopathy is from all-trans retinoic acid. In some embodiments, the cardiomyopathy is from an anti-retro viral agent. In some embodiments, the cardiomyopathy is from a phenothiazine. In some embodiments, the cardiomyopathy is chosen from one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five or twenty six of the non-familial causes. In some embodiments, the myocarditis is infective, toxic or immune myocarditis. In some embodiments, the myocarditis is infective myocarditis. In some embodiments, the myocarditis is toxic myocarditis. In some embodiments, the myocarditis is immune myocarditis. In some embodiments, the eosinophilic disease is churg strauss syndrome. In some embodiments, the nutritional imbalance is chosen from a thymine nutritional imbalance, a carnitine nutritional imbalance, a selenium nutritional imbalance, hypophosphataemia, or hypocalcaemia. In some embodiments, the nutritional imbalance is a thymine nutritional imbalance. In some embodiments, the nutritional imbalance is a carnitine nutritional imbalance. In some embodiments, the nutritional imbalance is a selenium nutritional imbalance. In some embodiments, the nutritional imbalance is hypophosphataemia. In some embodiments, the nutritional imbalance is hypocalcaemia. In some embodiments, the amyloid dysfunction is AL/prealbumin.

In some embodiments, the cardiomyopathy is from drugs chosen from serotonin, methysergide, ergotamine, mecurial agents, or busulfan. In some embodiments, the drug is serotonin. In some embodiments, the drug is methysergide. In some embodiments, the drug is ergotamine. In some embodiments, the drug is a mecurial agent. In some embodiments, the drug is busulfan.

In some embodiments, the antineoplastic drugs are chosen from anthracyclines, antimetabolites, alkylating agents, taxol, hypomethylating agents, monoclonal antibodies, tyrosine kinase inhibitors, and immunomodulating agents.

In some embodiments, the psychiatric drugs are chosen from clozapine, olanzapine, chloropromazine, risperidone, lithium methylphenidate, and tricyclic antidepressants.

In some embodiments, the patient suffers from a metabolic cardiomyopathy, in particular, from an age-related metabolic cardiomyopathy.

In some embodiments, the patient suffers from a metabolic cardiomyopathy. In some embodiments, the patient suffers from an age-related metabolic cardiomyopathy.

As used herein, the term “metabolic cardiomyopathy” relates to a cardiomyopathy associated with metabolic syndrome, such as obesity, diabetes/insulin resistance, hypertension and dyslipidemia. In particular, “metabolic cardiomyopathy” refers to cardiac consequences of metabolic syndrome such as atherosclerosis, coronary heart disease, obesity-associated heart disease, insulin resistance-associated heart disease, hypertensive heart disease, cardiac remodeling, heart failure and cardiometabolic diseases. As used herein, the term “age related metabolic cardiomyopathy” relates to any metabolic cardiomyopathy which has a factor of its etiology the age of the patient.

In some embodiments, the patient suffers from heart failure, in particular heart failure with preserved ejection fraction. In some embodiments, the patient suffers from heart failure. In some embodiments, the patient suffers from heart failure with preserved ejection fraction. In some embodiments, the patient suffers from myocardial infarction.

As used herein, the term “heart failure” or “HF” has its general meaning in the art and embraces congestive heart failure and/or chronic heart failure. Functional classification of heart failure is generally done by the New York Heart Association Functional Classification. This classification stages the severity of heart failure into 4 classes (I-IV). The classes (I-IV) are: Class I: no limitation is experienced in any activities, there are no symptoms from ordinary activities; Class II: slight, mild limitation of activity, the patient is comfortable at rest or with mild exertion; Class III: marked limitation of any activity, the patient is comfortable only at rest; and Class IV: any physical activity brings on discomfort and symptoms occur at rest.

As used herein, the term “heart failure with preserved ejection fraction” has its general meaning in the art and refers to a complex syndrome characterized by heart failure (HF) signs and symptoms and a normal or near-normal left ventricular ejection fraction (LVEF). More specific diagnostic criteria include signs/symptoms of HF, objective evidence of diastolic dysfunction, disturbed left ventricular (LV) filling, structural heart disease, and elevated brain natriuretic peptides. Additional cardiac abnormalities can include subtle alterations of systolic function, impaired atrial function, chronotropic incompetence, or haemodynamic alterations, such as elevated pre-load volumes.

As used herein, the term “DBI” has its general meaning in the art and refers to the diazepam binding inhibitor, acyl-CoA binding protein encoding by the DBI gene (Gene ID: 1622). The term is also known as EP, ACBP, ACBD1 and CCK-RP. An exemplary amino acid sequence for DBI is represented by SEQ ID NO: 1.

>sp|P07108|ACBP_HUMAN Acyl-CoA-binding protein
OS = Homo sapiens OX = 9606 GN = DBI PE = 1
SV = 2
SEQ ID NO: 1
MSQAEFEKAAEEVRHLKTKPSDEEMLFIYGHYKQATVGDINTERPGMLD
FTGKAKWDAWNELKGTSKEDAMKAYINKVEELKKKYGI

As described herein, an agent that reduces the activity or expression of DBI includes an agent that binds to DBI, and thus disrupt the function of DBI when administered. The specific agent that is utilized can be substituted without departing from the present disclosure. Indeed, the present disclosure provides for the use of any agent that can bind to and inhibit the activity or expression of DBI (such as extracellular DBI) in order to treat a cardiac dysfunction described herein. Such agents can include small molecules, polypeptides, or other agents that bind to and inhibit the activity or expression of DBI. Without wishing to be bound by theory, such agents when administered can bind to and block interaction with a binding partner of DBI. For example, the agent can be provided in circulation in order to bind to extracellular DBI present in circulation. The extracellular DBI, when bound by the agent, is thus blocked from performing its biological function. For example, the extracellular DBI, when present in circulation and bound by an agent as described herein, can no longer repress an autophagic state in neighbouring cells. Furthermore, the extracellular DBI, when present in circulation and bound by an agent as described herein, may be prohibited from binding to a biological binding partner such as gamma-aminobutyric acid type A receptor (GABR). Without wishing to be bound by theory, disrupting interaction of extracellular DBI to a biological binding partner using an agent described herein results in the treatment of cardiac dysfunction described herein. Furthermore, such agents when bound to extracellular DBI may indirectly result in reduction in levels of extracellular DBI in circulation. For example, extracellular DBI levels can be elevated in response to extracellular release of intracellular ACBP (for example, due to starvation-induced autophagy), resulting in inhibition of autophagy by the extracellular DBI. Without wishing to be bound by theory, an agent as described herein that reduces extracellular release of intracellular ACPB (for example, through binding to and inhibiting the activity of extracellular DBI) thus indirectly reduces the levels of extracellular DBI in circulation by presenting further extracellular export of intracellular ACBP. Alternatively, an agent can directly reduce the expression of DBI (including extracellular DBI) by silencing the expression of the DBI gene. Such agents include, for example, siRNAs, endonucleases, antisense oligonucleotides or ribozymes as described herein.

Thus, while the present application provides exemplary agents that reduce the activity or expression of DBI, these agents are merely exemplary.

In some embodiments, the agent that reduces the activity or expression of DBI is an antibody or an aptamer directed against DBI. In some embodiments, the agent is an antibody directed against DBI. In some embodiments, the agent is an aptamer directed against DBI. In some embodiments, the agent that reduces the expression of DBI is an inhibitor of expression.

In some embodiments, the agent that inhibits the activity of DBI is an antibody directed against DBI. In some embodiments, the agent is thus a neutralizing anti-DBI monoclonal antibody. In some embodiments, the antibody is directed against a fragment of DBI consisting of amino acid residue 43 to the amino acid residue 50 of SEQ ID NO: 1.

In some embodiments, the antibody is a monoclonal chimeric antibody, a monoclonal humanized antibody, or a monoclonal human antibody.

Any anti-DBI antibody that inhibits the activity of DBI (e.g., extracellular DBI) is suitable for use in the methods and compositions described herein. Such anti-DBI antibodies are commercially available and described in literature, and the sequences of such antibodies are known or can be derived. For instance, an antibody that inhibits the activity of DBI (e.g., extracellular DBI) and suitable for use as described herein has at least 80%, at least 85%, at least 90%, at least 95%, or has 100% sequence identity to a polypeptide sequence of an antibody selected from the group consisting of: ab231910 (Rabbit polyclonal, abcam); ab232760 (Rabbit polyclonal, abcam); ab16871 (Rabbit polyclonal, abcam); sc-30190 (Rabbit polyclonal, Santa Cruz Biotechnology); FNab02256 (Rabbit polyclonal, Wuhan Fine Biotech Co); PA5-89139 (Rabbit polyclonal, Invitrogen); OTI4A8 (Mouse monoclonal, OriGene); OTI6E12 (Mouse monoclonal, OriGene), mAb 7A (Mouse monoclonal, Fred Hutch Antibody Technology); Abcam (catalogue no. ab16871; RRID: AB_302557); DBI human or mouse FL-87 monoclonal antibodies from Santa Cruz (catalogue number sc-30190; RRID: AB_2211046); DBI human C-9 polyclonal antibodies from Santa Cruz (catalogue number sc-376853; RRID: AB_2722761) DBI mouse polyclonal antibodies from Abcam (catalogue number ab231910); DBI mouse 7a monoclonal antibodies from Fred Hutch Antibody Technology, DBI polyclonal antibodies from Invitrogen (catalogue numbers PA5-89139, PA5-79138, PA5-40659, PA5-102751, PA5-84066, PA5-76729 and PA5-92426); DBI monoclonal antibodies from OriGene (catalogue numbers CF813069, CF813070, CF813117, TA813069, TA81370, and TA813117); DBI polyclonal antibodies from Proteintech (catalogue number 14490-1-AP); DBI polyclonal antibodies from Abnova (catalogue number H00001622-D01P); or more than one of the foregoing.

Antibodies described herein may be obtained commercially or synthesized through any suitable method. For example, anti-DBI human monoclonal antibodies may be synthesized using peptides derived from the full length human ACBP and the phage display technology. In some embodiments, antibodies described herein are mutated antibodies. In some embodiments, antibodies described herein are selected based on favorable kinetic parameters, such as specificity for or affinity against human ACBP. Specificities of antibodies described herein can be validated by western blot, immunofluorescence and flow cytometry on human ACBP/DBI knock out cell lines.

As used herein the term “antibody” and “immunoglobulin” have the same meaning, and will be used equally in the present disclosure. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chains, lambda (1) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered. This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the term “neutralizing anti-DBI monoclonal antibody” refers to a monoclonal antibody having specificity for DBI and that inhibits or reduces the activity of DBI. Whether an antibody is a neutralizing antibody can be determined by in vitro assays described in the example section disclosed herein. Typically, the neutralizing antibody of the present disclosure inhibits the activity of DBI by at least 50%, 60%, 70%, 80%, 90%, 95%, 99%, or 100%.

As used herein, the terms “monoclonal antibody,” “monoclonal Ab,” “monoclonal antibody composition” and “mAb” or the like, refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

In some embodiments, the antibody is directed against the fragment consisting in the amino acid sequence ranging from the amino acid residue at position 43 to the amino acid residue at position 50 in SEQ ID NO: 1 (i.e., the octapeptide or OP).

In some embodiments, the antibody of the present disclosure is a chimeric antibody, typically a chimeric mouse/human antibody.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance.

In some embodiments, the antibody is a humanized antibody.

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin.

In some embodiments, the antibody is a human antibody.

As used herein the term “human antibody” as used herein, is intended to include antibodies having variable and constant regions derived from human immunoglobulin sequences. The human antibodies of the present disclosure may include amino acid residues not encoded by human immunoglobulin sequences (e.g., mutations introduced by random or site-specific mutagenesis in vitro or by somatic mutation in vivo). However, the term “human antibody” as used herein is not intended to include antibodies in which CDR sequences derived from the germline of another mammalian species, such as a mouse, have been grafted onto human framework sequences.

In some embodiments, the neutralizing antibody of the present disclosure does not mediate antibody-dependent cell-mediated cytotoxicity and thus does not comprise an Fc portion that induces antibody dependent cellular cytotoxicity (ADCC). In some embodiments, the neutralizing antibody does not comprise an Fc domain capable of substantially binding to a FcgRIIIA (CD16) polypeptide. In some embodiments, the neutralizing antibody lacks an Fc domain (e.g., lacks a CH2 and/or CH3 domain) or comprises an Fc domain of IgG2 or IgG4 isotype. In some embodiments, the neutralizing antibody consists of or comprises a Fab, Fab′, Fab′-SH, F (ab′) 2, Fv, a diabody, single-chain antibody fragment, or a multispecific antibody comprising multiple different antibody fragments. In some embodiments, the neutralizing antibody is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC).

In some embodiments, the agent that inhibits the activity of DBI is an aptamer directed against DBI. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

In some embodiments, the agent that inhibits the expression of DBI is an inhibitor of expression. In a preferred embodiment of the present disclosure, said inhibitor of gene expression is an siRNA, an endonuclease, an antisense oligonucleotide or a ribozyme. In some embodiments, the agent that reduces the expression of DBI is an inhibitor of expression. In some embodiments, the inhibitor of expression is an siRNA, an endonuclease, an antisense oligonucleotide or a ribozyme. In some embodiments, the inhibitor of expression is an siRNA. In some embodiments, the inhibitor of expression is an endonuclease. In some embodiments, the inhibitor of expression is an antisense oligonucleotide. In some embodiments, the inhibitor of expression is a ribozyme.

In some embodiments, the agent that inhibits the activity of DBI consists of a vaccine composition suitable for eliciting neutralizing autoantibodies against DBI when administered to the subject.

In some embodiments, the agent that reduces the activity of DBI is a vaccine composition suitable for eliciting neutralizing autoantibodies against DBI when administered to the patient. In some embodiments, the vaccine composition comprises a polypeptide antigen comprising (i) an amino acid sequence having at least 80% identity with SEQ ID NO: 1; (ii) an amino acid sequence having at least 80% identity to a polypeptide fragment consisting of the amino acid sequence ranging from amino acid residue 17 to amino acid residue 50 of SEQ ID NO: 1; (iii) an amino acid sequence having at least 80% identity to a polypeptide fragment consisting of the amino acid sequence ranging from amino acid residue 33 to amino acid residue 50 of SEQ ID NO: 1; or (iv) an amino acid sequence having at least 80% identity to a polypeptide fragment consisting of the amino acid sequence ranging from amino acid residue 43 to amino acid residue 50 of SEQ ID NO: 1. In some embodiments, the amino acid sequence has at least 80% identity with SEQ ID NO: 1. In some embodiments, the amino acid sequence has at least 80% identity, at least 80% identity, at least 85% identity, at least 90% identity, at least 95% identity, or at least 98% identity with SEQ ID NO: 1. In some embodiments, the polypeptide antigen comprises an amino acid sequence having at least 80% identity to a polypeptide fragment consisting of the amino acid sequence ranging from amino acid residue 17 to amino acid residue 50 of SEQ ID NO: 1. In some embodiments, the polypeptide antigen comprises an amino acid sequence having at least 80% identity to a polypeptide fragment consisting of the amino acid sequence ranging from amino acid residue 33 to amino acid residue 50 of SEQ ID NO: 1. In some embodiments, the polypeptide antigen comprises an amino acid sequence having at least 80% identity to a polypeptide fragment consisting of the amino acid sequence ranging from amino acid residue 43 to amino acid residue 50 of SEQ ID NO: 1. In some embodiments, the amino acid sequence has at least 80% identity, at least 85% identity, at least 90% identity, at least 95% identity, or at least 98% identity with any of the foregoing polypeptide fragments of SEQ ID NO: 1.

For the purpose of the present disclosure, the term “vaccine composition” is intended to mean a composition which can be administered to humans or to animals in order to induce an immune system response; this immune system response can result in the production of antibodies against DBI. Typically, the vaccine composition comprises at least one antigen derived from DBI. As used herein the term “antigen” refers to a molecule capable of being specifically bound by an antibody or by a T cell receptor (TCR) if processed and presented by NMC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes). In some embodiments, the antigen consists of a polypeptide comprising an amino acid sequence having at least 80% of identity with the sequence of SEQ ID NO: 1 or a fragment thereof (e.g., an epitope). In some embodiments, the antigen consists in a polypeptide comprising (i) an amino acid sequence having at least 80% of identity with SEQ ID NO: 1 or (ii) an amino acid sequence having at least 80% of identity with the amino acid sequence ranging from the amino acid residue at position 17 to the amino acid residue at position 50 in SEQ ID NO: 1; or (iii) an amino acid sequence having at least 80% of identity with the amino acid sequence ranging from the amino acid residue at position 33 to the amino acid residue at position 50 in SEQ ID NO: 1; or (iv) an amino acid sequence having at least 80% of identity with the amino acid sequence ranging from the amino acid residue at position 43 to the amino acid residue at position 50 in SEQ ID NO: 1. In some embodiments, the polypeptide is conjugated to a carrier protein which is generally sufficiently foreign to elicit a strong immune response to the vaccine. Illustrative carrier proteins are inherently highly immunogenic. Both bovine serum albumin (BSA) and keyhole limpet hemocyanin (KLH) have commonly been used as carriers in the development of conjugate vaccines when experimenting with animals and are contemplated herein as carrier proteins. Proteins which have been used in the preparation of therapeutic conjugate vaccines include, but are not limited to, a number of toxins of pathogenic bacteria and their toxoids. Suitable carrier molecules are numerous and include, but are not limited to: Bacterial toxins or products, for example, cholera toxin B-(CTB), diphtheria toxin, tetanus toxoid, pertussis toxin, filamentous hemagglutinin, shiga toxin and pseudomonas exotoxin; Lectins, for example, ricin-B subunit, abrin and sweet pea lectin; Sub virals, for example, retrovirus nucleoprotein (retro NP), rabies ribonucleoprotein (rabies RNP), plant viruses (e.g., TMV, cow pea and cauliflower mosaic viruses), vesicular stomatitis virus-nucleocapsid protein (VSV-N), poxvirus vectors and Semliki forest virus vectors; Artificial vehicles, for example, multiantigenic peptides (MAP), microspheres; Yeast virus-like particles (VLPs); Malarial protein antigen; and others such as proteins and peptides as well as any modifications, derivatives or analogs of the foregoing. Other useful carriers include those with the ability to enhance a mucosal response, more particularly, LTB family of bacterial toxins, retrovirus nucleoprotein (retro NP), rabies ribonucleoprotein (rabies RNP), vesicular stomatitis virus-nucleocapsid protein (VSV-N), and recombinant pox virus subunits.

DBI is known to be released from cells through an unconventional (Golgi-independent) pathway, in an autophagy-dependent fashion, as was first demonstrated for several fungal species. In some embodiments of the present disclosure, the agent that reduces the activity or expression of DBI reduces the activity or expression of extracellular DBI. In some embodiments, the agent reduces the activity of extracellular DBI by binding to extracellular DBI. In some embodiments, the binding to the extracellular DBI disrupts binding of the extracellular DBI to a binding partner naturally present in a human cell. In some embodiments, the binding partner is a gamma-aminobutyric acid type A receptor (GABR). In some embodiments, the agent as disclosed herein binds to a surface of the extracellular DBI that is responsible for binding of the extracellular DBI to the GABR. In some embodiments, the agent reduces levels of extracellular DBI in circulation. In some embodiments, the agent reduces the activity of extracellular DBI, thereby resulting in induction of autophagy. In some embodiments, the induction of autophagy reduces levels of extracellular DBI in circulation. In some embodiments, the agent that reduces the levels of extracellular DBI in circulation does not reduce levels of intracellular DBI when administered to the patient.

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required for achieving the desired therapeutic effect and gradually increasing the dosage until the desired effect is achieved. In general, a suitable dose of a composition of the present disclosure will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the patient's size, the severity of the patient's symptoms, and the particular composition or route of administration selected. An exemplary, non-limiting range for a therapeutically effective amount of a drug of the present disclosure is about 0.1-100 mg/kg, such as about 0.1-50 mg/kg, for example about 0.1-20 mg/kg, such as about 0.1-10 mg/kg, for instance about 0.5 mg/kg, about such as 0.3 mg/kg, about 1 mg/kg, about 3 mg/kg, about 5 mg/kg or about 8 mg/kg. An exemplary, non-limiting range for a therapeutically effective amount of a drug of the present disclosure is 0.02-100 mg/kg, such as about 0.02-30 mg/kg, such as about 0.05-10 mg/kg or 0.1-3 mg/kg, for example about 0.5-2 mg/kg.

Typically, the agent that inhibits the activity or expression of DBI is administered to the patient in the form of a pharmaceutical composition which comprises a pharmaceutically acceptable carrier.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present disclosure may be administered orally, parenterally, by inhalation spray, topically, rectally, nasally, buccally, vaginally or via an implanted reservoir. The administrations of the present disclosure include subcutaneous, intravenous, intramuscular, intra-articular, intra-synovial, intrasternal, intrathecal, intrahepatic, intralesional and intracranial injection or infusion techniques. Sterile injectable forms of the compositions of this disclosure may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solids, liquids, or other dosage forms may also be used for the purposes of formulation. The compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the agent that inhibits the activity or expression of DBI is combined with emulsifying and suspending agents. If desired, certain sweetening, flavoring or coloring agents may also be added. Alternatively, the compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, the anti-DBI agent of the present disclosure can be used to treat an ischemic cardiac condition. As described herein, extracellular DBI levels in circulation are increased in response to stress induced on the heart, such as an ischemic event that results in limited oxygen availability. Without wishing to be bound by theory, the presence of the increased levels of extracellular DBI in circulation after the ischemic event results in the progression of cardiac dysfunction in a patient. By neutralizing the levels of extracellular DBI using an agent described herein, the present disclosure provides for treatment of conditions associated with the ischemic event. For example, administration of an anti-DBI agent as described herein can be used to treat ischemic cardiomyopathy, myocardial ischemia (with or without infarction) and any other cardiac conditions associated with limited oxygen availability or reduced cell viability. Furthermore, because the levels of extracellular DBI increase as a result of the ischemic event, the ischemic event itself can be diagnosed in the patient by measuring an increase in extracellular DBI in the subject (such as by performing an immunoassay on a sample obtained from the subject). Once diagnosed, the ischemic event can then be treated by administration of the anti-DBI agent. Thus, some embodiments of the present disclosure provide for a method of detecting and treating ischemia (such as myocardial ischemia) in a patient, the method comprising: (a) measuring a level of extracellular diazepam binding inhibitor (DBI) in a plasma sample obtained from the patient, wherein the level of extracellular DBI is determined in an in vitro immunoassay; (b) comparing the level of extracellular DBI in the patient to a reference level of extracellular DBI from a healthy subject; (c) diagnosing the patient as having ischemia when the level extracellular DBI in the patient is higher than the reference level; and (d) administering a therapeutically effective amount of an agent that reduces the activity or expression of extracellular DBI to the patient as described herein, thereby treating the ischemia.

Some embodiments of the present disclosure provide for a composition for use in treating cardiac dysfunction in a patient in need thereof, wherein the composition comprises a therapeutically effective amount of an agent that reduces the activity or expression of diazepam binding inhibitor (DBI) as described herein.

In some embodiments, the composition for use is used for improving cardiac remodeling, cardiac fibrosis, impairment of systolic function, or impairment of diastolic dysfunction. In some embodiments, the composition for use is used for improving cardiac remodeling. In some embodiments, the composition for use is used for improving cardiac fibrosis. In some embodiments, the composition for use is used for improving impairment of systolic function. In some embodiments, the composition for use is used for improving impairment of diastolic dysfunction. In some embodiments, the composition for use is used for treating cardiomyopathy. In some embodiments, the cardiomyopathy is ischemic cardiomyopathy. In some embodiments, the composition for use is used for treating heart failure. In some embodiments, the composition for use is used for treating myocardial infarction.

The present disclosure will be further illustrated by the accompanying figures and examples. However, these examples and figures should not be interpreted in any way as limiting the scope of the present disclosure.

### EXAMPLES

Acyl coenzyme A binding protein (ACBP), which is encoded by diazepam-binding inhibitor (DBI), is a leaderless polypeptide that can be secreted in an unconventional fashion during the activation of autophagy. Applicants have found that circulating ACBP/DBI levels increase with aging and obesity. However, the link between ACBP/DBI levels and cardiac disease has thus far not been established. As such, Applicant described herein that inhibition of ACBP/DBI reduces cardiac disease (including cardiac disease associated with aging and other diseases). Applicants used an animal model of anthracycline-induced cardiomyocyte senescence to determine whether ACBP is causally involved in cardiac disease. Heart failure with preserved ejection fraction (HFpEF) is currently the predominant form of heart failure and the leading cause of hospitalization in the elderly. However, limited understanding of the underlying mechanisms of HFpEF has led to a longstanding absence of evidence-based therapies. In this respect, growing epidemiological and experimental evidence suggests that metabolic dysfunction might drive the pathogenesis of HFpEF. Because the majority of HFpEF patients are obese or diabetic, Applicants hypothesized that ACBP/DBI, which can act as a metabolic effector of the heart and peripheral organs, might be therapeutically targeted for the treatment of HFpEF.

### Methods

Cardiac toxicity experiments: Eight-week-old C57Bl/6J female mice were purchased from Envigo (Gannat, France). Following 2 weeks of acclimatization, mice were randomized (1:1) to receive either mouse monoclonal anti-ACBP-neutralizing antibody (5 mg/kg body weight, injected intraperitoneally once per week) or mouse isotype IgG (negative control; 5 mg/kg body weight). Each group was further randomized (1:1) to receive a weekly intraperitoneal injection of saline or doxorubicin (5 mg/kg body weight; cumulative dose of 20 mg/kg body weight over 4 weeks) (Sigma, #MKCM8540). Of note, anti-ACBP was always administered 24 hours prior to doxorubicin injection. All mice used in this study were housed in a temperature-controlled environment with 12 h light/dark cycles and ad libitum access to food and water.

Immunohistochemistry of p21: On the day of euthanasia, left ventricles were briefly rinsed with PBS and fixed for 16-24 h in a 4% formaldehyde solution (F8775, Sigma, diluted in PBS, pH=7.4). After fixation, tissues were transferred to 70% ethanol until inclusion in paraffin. Paraffin-embedded tissue were cut to 2-3 μm sections, air dried and let to dry completely at 60° C. overnight. Cell Conditioning 1 buffer (64/457,5001, Roche) was used for antigen retrieval, and casein (ref: 760-219, Roche) was used as blocking agent. The antibody directed against p21 (clone HUGO 291H/B5, CNIO) was diluted 1:50 in EnVision FLEX Antibody Diluent (K800621, Dako-Agilent). Staining was performed on the Ventana discovery XT platform for 60 minutes (ready-to-use). Secondary staining was done with the OmniMap anti-rat HRP (760-4457, Roche) and the ChomoMap DAB kit (760-159, Roche) was used for revelation. Slides were counterstained with hematoxylin (S202084, Dako-Agilent) and mounted by means of the Dako CoverStainer with toluene-free mounting medium (CS705, Dako-Agilent). The specificity of the staining was assessed in each organ by using rat IgG isotype control (6-001-F, R&D Systems) as primary antibody.

Quantification of the p21 staining was done on the QuPath analysis software after blinding the samples names. Five 250 μm-wide squares were chosen to be representative of longitudinally cut cardiomyocytes, and five others were chosen in the transversally-cut regions. Non-cardiomyocytes regions were excluded from the area of analysis and, after color deconvolution, a threshold was applied to automatically quantify the p21+ cardiomyocytes area. The average value in the 10 regions of interest was plotted for each animal, and analyzed by two-way ANOVA to determine the effect of doxorubicin treatment, ACBP/DBI neutralization, and their interaction.

HFpEF model: Eight-week-old C57Bl/6J male mice were purchased from Envigo (Gannat, France). Following 2 weeks of acclimatization, mice were randomized (1:1) to receive either HFD (Safe, #260 HF) and L-NAME (0.5 g/l in the drinking water) or a regular diet (Safe, #A04) and normal drinking water. Each group was further randomized (1:1) to receive mouse monoclonal anti-ACBP-neutralizing antibody (5 mg/kg body weight, injected intraperitoneally once per week) or mouse isotype IgG (negative control; 5 mg/kg body weight). As for conditional deletion of Acbp, Acbp^{-/-} mice were generated by crossing B6.Cg-Tg(UBC-cre/ERT2)1Ejb/1J mice (Jackson Laboratory, Bar Harbor, ME, USA) and Acbp^{fl/fl} mice (OZgene, Bentley, WA, USA). Genotype was verified by PCR. At the age of 8 weeks, Cre recombinase was activated by tamoxifen injections (75 mg/kg body weight IP, daily for 5 days). HFD+L-NAME treatment was initiated in Acbp^{-/-} and their Acbp^{+/+} littermates 8 weeks after the last tamoxifen injection, thereby avoiding transient tamoxifen-related cardiomyopathy. After 8 weeks of HFD+L-NAME treatment, all mice were assessed using non-invasive echocardiography (Vevo3100, Fujifilm VisualSonics Inc., Canada). Animal experiments were performed according to the European ethical regulations (Directive 2010/63/EU) and were approved by the Animal Experimental Ethics Committee in France (protocol #35132).

Echocardiography. Mice were lightly anesthetized (5% for induction; 1.5% isoflurane for maintenance) and body temperature was kept at 37° C. using a temperature-controlled heating platform. Mice were placed in a supine position with their limbs in direct contact with non-invasive electrocardiogram leads for heart rate assessment. Pre-warmed ultrasound transmission gel was spread on a shaved chest to obtain cardiac tracings in the parasternal long axis using high-resolution 55 MHz linear-array probe. M-mode tracings were used to evaluate cardiac walls thickness and internal left ventricular dimensions at the level of the papillary muscles during systole and diastole. Ventricular volumes and myocardial mass were estimated using Teichholtz and Troy formulas, respectively. Ejection fraction was determined to assess systolic function, while diastolic function was evaluated using the ratio between peak early-filling velocity of transmitral flow (E) and the corresponding mitral valve annulus velocity (e′) determined using pulsed-wave and tissue Doppler imaging, respectively. Generally, at least 3 stable cardiac cycles were averaged to obtain the reported parameters.

### Results

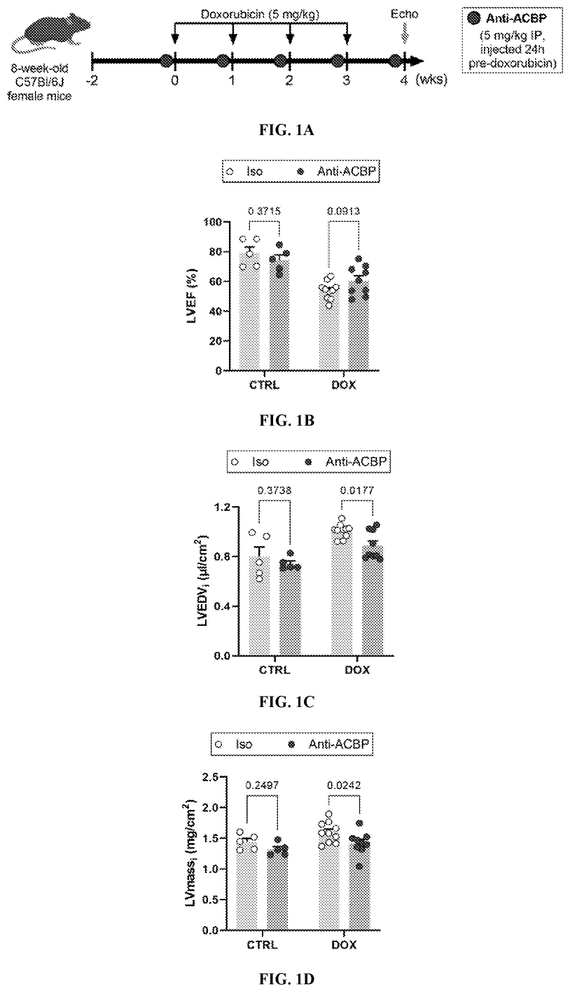

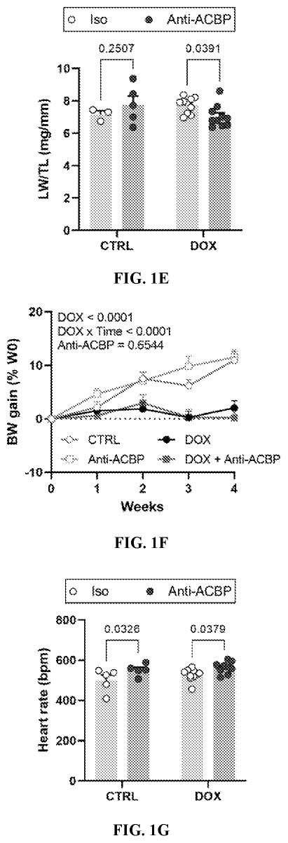

ACBP/DBI neutralization reduces anthracycline-accelerated cardiac aging. An animal model of anthracycline-induced cardiomyocyte senescence was used to determine whether ACBP is causally involved in cardiac disease (FIG. 1A). Mice were subjected to chronic doxorubicin (DOX) treatment (cumulative dose: 20 mg/kg body weight, injected intraperitoneally over 4 weeks). DOX treatment clearly reduced left ventricular ejection fraction (p<0:001, FIG. 1B), thereby causing ventricular dilation (P=0.001; FIG. 1C). By contrast, treatment of DOX mice with a murine monoclonal ACBP-neutralizing antibody (Anti-ACBP; 5 mg/kg body weight, injected IP weekly) partially preserved cardiac function, as suggested by a significant reduction in left ventricular dilation, despite an unaltered ejection fraction (FIGS. 1B-1C). Anti-ACBP-treated mice also exhibited lower left ventricular mass index and tibia length-normalized lung weight (FIGS. 1D-1E), indicating reduced cardiac remodelling and lung congestion, respectively. Of note, doxorubicin-induced suppression of body weight gain was not affected by anti-ACBP (FIG. 1F), whereas anti-ACBP appeared to increase heart rate, irrespective of DOX treatment (FIG. 1G).

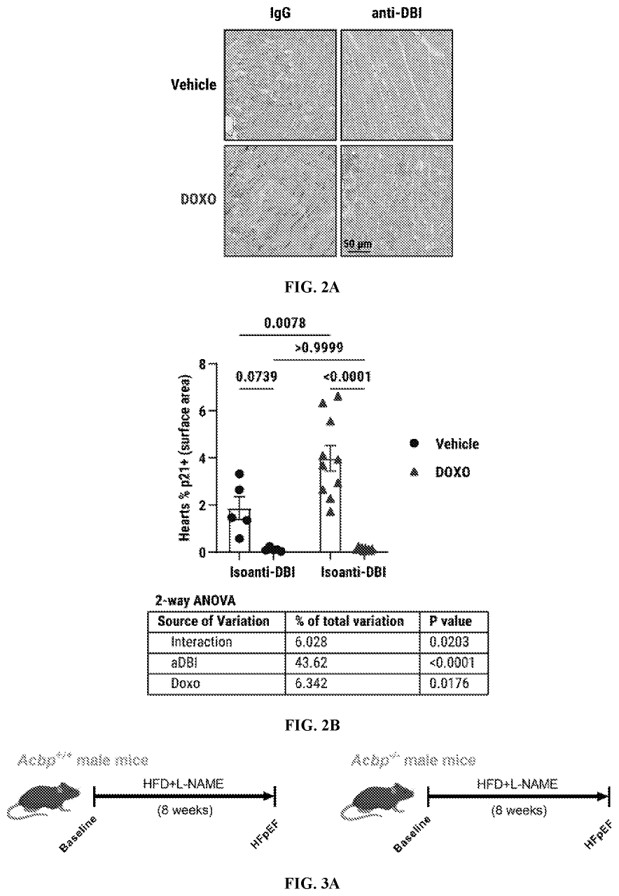

In parallel, Applicant sought to understand the impact of the antibody on the induction of senescence by doxorubicin by studying one of the most widely used markers, the nuclear p21 protein. DOX damage increased the presence of p21 staining in cardiomyocytes, a phenomenon that was completely abrogated by concomitant treatment with anti-ACBP (FIGS. 2A and 2B). Taken together, ACBP neutralization reduces the cardiotoxicity of elevated anthracycline doses and shows a senolytic activity.

### ACBP/DBI Neutralization Reduces HFpEF

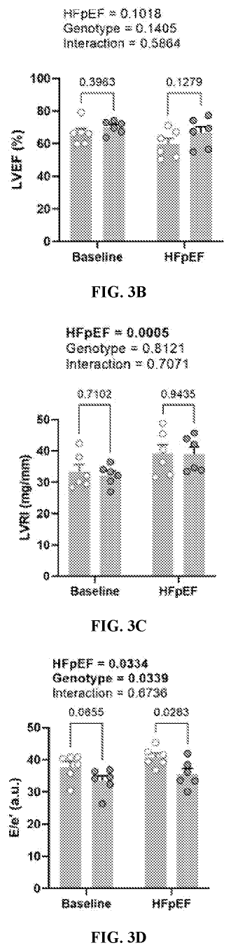

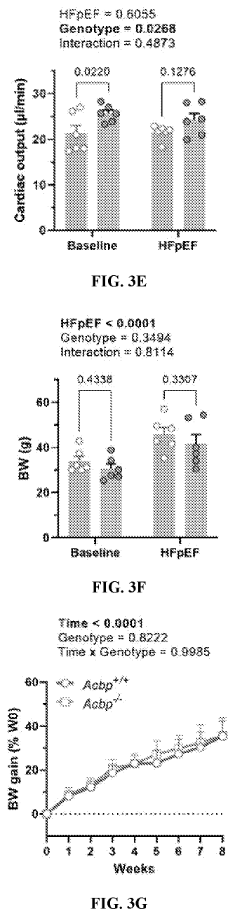

To examine whether ACBP neutralization improves HFpEF in mice, Acbp knockout mice (Acbp^{-/-}) were generated, in which Acbp was conditionally depleted upon tamoxifen administration. Male Acbp^{-/-} and their wild type littermates (Acbp^{+/+}) were subjected to a ‘two-hit’ HFpEF model using high-fat diet (HFD) and the nitric oxide synthase inhibitor N[ω]-nitro-1-arginine methyl ester (L-NAME) (FIG. 3A). After 8 weeks, cardiac structure and function were comprehensively assessed using echocardiography. HFD+L-NAME feeding did not alter ejection fraction (FIG. 3B), but effectively induced cardiac remodelling and diastolic dysfunction, as indicated by a higher left ventricular remodelling index (LVRI) and an increased E/e′ (peak early Doppler transmitral flow velocity-to-myocardial tissue Doppler velocity) ratio (FIGS. 3C-3D). Although ejection fraction and LVRI were similar in Acbp^{+/+} and Acbp^{-/-} mice (FIGS. 3B-3C), Acbp^{-/-} mice exhibited significantly improved E/e′ ratio (FIG. 3D), denoting improved diastolic function—the cardinal sign of HFpEF. ACBP-depleted mice also showed superior cardiac output as compared to their ACBP-expressing littermates (FIG. 3E), which occurred in absence of body weight alterations (FIGS. 3F-3G), thus, excluding potential secondary cardiac effects to reduced adiposity.

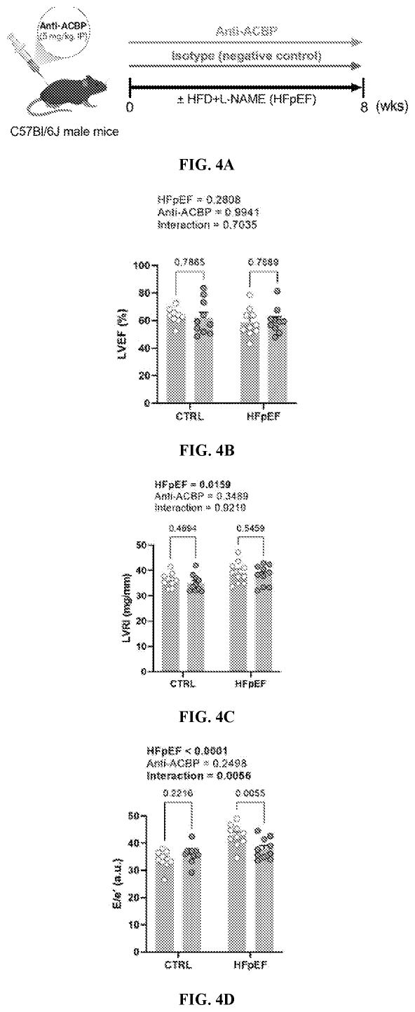

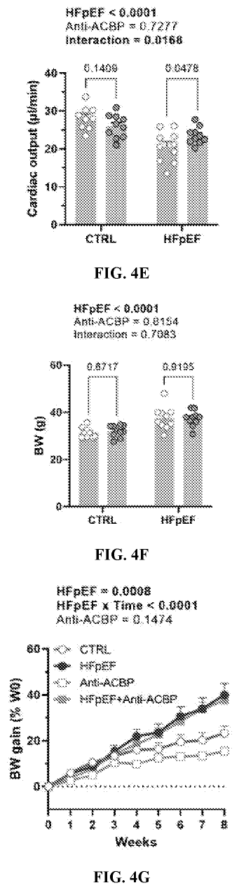

In an effort to improve the translational potential of these findings, the anti-HFpEF effects of ACBP neutralization were analyzed using a murine monoclonal ACBP-neutralizing antibody (anti-ACBP). For this, wild type C67Bl/6J male mice were subjected to the same protocol using HFD and L-NAME with a subset of mice receiving anti-ACBP (5 mg/kg body weight, injected IP) once a week (FIG. 4A). In line with Acbp knockout, anti-ACBP did not alter EF or LVRI, but remarkably improved diastolic dysfunction and cardiac output without affecting relative or absolute body weight gain (FIGS. 4B-4G).

Here, Applicants show that neutralization of ACBP/DBI reduces the deleterious effect of the anthracycline doxorubicin on cardiac function. Hence inhibition of ACBP/DBI or of its receptor may be useful for the prevention or treatment of anthracycline-induced heart failure or for antagonizing the cardiotoxic effects of other pharmacological agents including but not limited to direct and indirect sympathomimetics, beta-blockers, anticholinergics, cholinomimetics, adenosine receptor agonist, Ca² channel blockers, Na⁺/K⁺ ATPase inhibitors, lysosomotropic agents, anticancer chemotherapeutics (such as cytarabine, cyclophosphamide, daunorubicin, docetaxel, epirubicin, paclitaxel, treosulfan and other anthracyclines, alkylating agents and taxanes), HER2-targeting antibodies (such as adotrastuzumab trastuzumab, pertuzumab and others), tyrosine kinase inhibitors (such as dobrafenib, lapatinib, sorafenib, sunitinib, ponatinib and others), proteasome inhibitors (such as bortezomib, karfilzomib and others), non-steroidal anti-inflammatory agents, ethanol, nicotine and smoking. Since anthracycline-induced heart failure and anthracycline-induced senescence are a model of accelerated cardiac aging, it is also plausible to use ACBP/DBI inhibition or neutralization as a method to prevent or treat aging of the cardiovascular system.

Applicants surprising found that elevated DBI levels does not merely correlate to cardiac dysfunction, but inhibiting DBI activity has a direct effect on cardiac dysfunction. Applicants also found that genetic and pharmacological interventions to deplete ACBP efficiently improve cardiac dysfunction associated with experimental HFpEF, a condition associated with aging, hypertension and obesity.

1. A method of treating cardiac dysfunction in a patient in need thereof, the method comprising administering a therapeutically effective amount of an agent that reduces the activity or expression of diazepam binding inhibitor (DBI) to the patient.

2. The method of claim 1, wherein the cardiac dysfunction comprises ischemic cardiomyopathy, myocardial ischemia, myocardial infarction, or any cardiac condition associated with limited oxygen availability or reduced cell viability.

3. The method of claim 1, wherein the agent improves cardiac remodeling, cardiac fibrosis, impairment of systolic function, or impairment of diastolic dysfunction, each as compared to prior to the administering.

4. The method of claim 1, wherein the patient is an elderly patient, an obese patient, or a patient who exhibits one or more risk factors for cardiac dysfunction, wherein the one or more risk factors for cardiac dysfunction is chosen from age, alcohol consumption, cigarette smoking, metabolic syndrome, obesity, diabetes/insulin resistance, hypertension, dyslipidemia, liver disease and chronic kidney disease.

5. (canceled)

6. The method of claim 1, wherein the patient suffers from a cardiomyopathy.

7. The method of claim 6, wherein the cardiomyopathy is dilated cardiomyopathy, metabolic cardiomyopathy, ischemic cardiomyopathy, hypertrophic cardiomyopathy, non-obstructive cardiomyopathy, restrictive cardiomyopathy, left ventricular non-compaction, or arrhythmogenic right ventricular cardiomyopathy, and/or the cardiomyopathy is from one or more of the following non familial causes: obesity, a diabetic mother, athletic training, myocarditis, Kawasaki disease, an eosinophilic disease, viral persistence, pregnancy, endocrine dysfunction, a nutritional imbalance, alcohol, tachycardiomyopathy, inflammation, amyloid dysfunction, scleroderma, endomyocardial fibrosis, hypereosinophilic syndrome, a drug, carcinoid heart disease, a metastatic cancer, an antineoplastic drug, a psychiatric drug, chloroquine, all-trans retinoic acid, an anti-retro viral agent, and a phenothiazine.

8. (canceled)

9. (canceled)

10. (canceled)

11. (canceled)

12. (canceled)

13. (canceled)

14. (canceled)

15. (canceled)

16. (canceled)

17. The method of claim 1, wherein the patient suffers from heart failure.

18. The method of claim 17, wherein the heart failure is a heart failure with preserved ejection fraction.

19. The method of claim 1, wherein the patient suffers from myocardial infarction.

20. The method according to claim 1, wherein the agent that reduces the activity of DBI is an antibody or an aptamer directed against DBI.

21. The method of claim 20, wherein the antibody is directed against a fragment of DBI comprising amino acid residue 43 to amino acid residue 50 of SEQ ID NO: 1.

22. The method of claim 20, wherein the antibody is a monoclonal chimeric antibody, a monoclonal humanized antibody, or a monoclonal human antibody.

23. The method according to claim 1, wherein the agent that reduces the expression of DBI is an inhibitor of expression selected from an siRNA, an endonuclease, an antisense oligonucleotide or a ribozyme.

24. (canceled)

25. The method according to claim 1, wherein the agent that reduces the activity of DBI is a vaccine composition suitable for eliciting neutralizing autoantibodies against DBI when administered to the patient.

26. The method of claim 25, wherein the vaccine composition comprises a polypeptide antigen comprising (i) an amino acid sequence having at least 80% identity with SEQ ID NO: 1; (ii) an amino acid sequence having at least 80% identity to a polypeptide fragment consisting of the amino acid sequence ranging from amino acid residue 17 to amino acid residue 50 of SEQ ID NO: 1; (iii) an amino acid sequence having at least 80% identity to a polypeptide fragment consisting of the amino acid sequence ranging from amino acid residue 33 to amino acid residue 50 of SEQ ID NO: 1; or (iv) an amino acid sequence having at least 80% identity to a polypeptide fragment consisting of the amino acid sequence ranging from amino acid residue 43 to amino acid residue 50 of SEQ ID NO: 1.

27. The method of claim 1, wherein the agent reduces the activity or expression of extracellular DBI.

28. (canceled)

29. The method of claim 27, wherein the agent reduces the activity of extracellular DBI by binding to extracellular DBI, thereby disrupting binding of the extracellular DBI to a binding partner naturally present in a human cell.

30. (canceled)

31. The method of claim 29, wherein the binding partner is a gamma-aminobutyric acid type A receptor (GABR).

32. (canceled)

33. (canceled)

34. (canceled)

35. The method of claim 33, wherein the agent that reduces the levels of extracellular DBI in circulation does not reduce levels of intracellular DBI when administered to the patient.

36. A method of detecting and treating myocardial ischemia in a patient, the method comprising:
(a) measuring a level of extracellular diazepam binding inhibitor (DBI) in a plasma sample obtained from the patient, wherein the level of extracellular DBI is determined in an in vitro immunoassay;
(b) comparing the level of extracellular DBI in the patient to a reference level of extracellular DBI from a healthy subject;
(c) diagnosing the patient as having myocardial ischemia when the level extracellular DBI in the patient is higher than the reference level; and
(d) administering a therapeutically effective amount of an agent that reduces the activity or expression of extracellular DBI to the patient, thereby treating the myocardial ischemia.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)
